(19) United States
(12) Patent Application Publication
(10) Pub. No.: US 2020/0289810 A1
(43) Pub. Date: Sep. 17, 2020

(54) DUAL LUMEN TUBING EXTENDING FROM BODILY INSERTED MEDICAL APPLIANCE AND COLOCATED INFUSION AND VACUUM PUMPS

(71) Applicant: VIADERM LLC, Plymouth, MI (US)

(72) Inventor: Allen B. Kantrowitz, Miami Beach, FL (US)

(73) Assignee: VIADERM LLC, Plymouth, MI (US)

(21) Appl. No.: 16/819,544

(22) Filed: Mar. 16, 2020

Related U.S. Application Data
(60) Provisional application No. 62/818,470, filed on Mar. 14, 2019.

(51) Int. Cl. A61M 39/08; A61M 39/10; A61M 39/02

(52) U.S. Cl. CPC A61M 39/08; A61M 39/105; A61M 2039/0202; A61M 2039/082; A61M 39/02

(57) ABSTRACT

An integrated multi-lumen tubing includes two or more lumens. A webbing is provided between each of said two or more lumens and holding the two or more lumens together as a ribbon. A set of terminations are provided at opposing ends of each of the two or more lumens. A sheath can also be provided surrounding the two or more lumens. A system for supply of intravenous fluid and vacuum to a patient includes the aforementioned integrated multi-lumen tubing. An intravenous bag or bottle is provided that is in fluid communication with an infusion pump. The integrated multi-lumen tubing connects the infusion pump and a vacuum pump to a percutaneous access device (PAD). A method of using said integrated multi-lumen tubing to delivery simultaneously two or more inputs into a subject in need thereof is also provided.

### RELATED APPLICATIONS

This application claims priority benefit of U.S. Provisional Application Ser. No. 62/818,470 filed 14 Mar. 2019; the contents of which are hereby incorporated by reference.

### FIELD OF THE INVENTION

The present invention in general relates to medical devices and systems and in particular to integrated multi-lumen tubing for a combination of intravenous infusion lines, vacuum lines, and in some instances monitoring lines for attachment to a percutaneous access device or long term implant.

### BACKGROUND OF THE INVENTION

In patients requiring long-term intravenous (IV) drug therapy, total parenteral nutrition, temporary access for kidney dialysis, or frequent blood testing, repeated access to a vein may be necessary over an extended period of time. Multiple needle sticks into a vein can be difficult, painful, and time-consuming. In such cases, a thin, flexible tube system known as a central venous catheter (also called a central line) may be inserted under the skin and into a large vein. This type of catheter may be safely and comfortably left in place for percutaneous access for days, weeks, or months. Additionally, peripherally inserted central catheters (PICC), skeletal guide wires, cardiac assist device lines, or other instruments may be kept in place for weeks or months with a precutaneous access device (PAD).

A common problem associated with implantation of a cutaneous access device (PAD) or other skin penetrating appliance is skin regeneration about the periphery of the appliance to form an immunoprotective seal against infection. New cell growth and maintenance is typically frustrated by the considerable mechanical forces exerted on the interfacial layer of cells. In order to facilitate skin regeneration about the exterior of the appliance, subject cells are often harvested and grown in culture onto appliance surfaces for several days prior to implantation in order to allow an interfacial cell layer to colonize appliance surfaces in advance of implantation. Unfortunately, cell culturing has met with limited acceptance owing to the need for a cell harvesting surgical procedure preceding the implantation procedure. Additionally, maintaining tissue culture integrity is also a complex and time-consuming task.

A related context in which cell growth is needed is wound healing, with DACRON® based random felt meshes have been used to promote cell regrowth in the vicinity of a wound, such felts have uncontrolled pore sizes that harbor bacterial growth pockets.

U.S. Pat. No. 7,704,225 to Kantrowitz solves many of these aforementioned problems by providing cell channeling contours, porous biodegradable polymers and the application of vacuum to promote cellular growth towards the surface the neck of a PAD. The facilitating of rapid cellular colonization of a PAD neck allows the subject to act as their own cell culture facility and as such affords more rapid stabilization of the PAD, and lower incidence of separation and infection.

FIG. 1 depicts a PAD generally at 100 as shown in U.S. application Ser. No. 13/416546 to Kantrowitz. A cap 102 is formed of a material such as silicone, a polymer or a metal and serves to keep debris from entering the device 100. Preferably, the cap 102 is remote from the surface of the epidermis E. The medical appliance 34 depicted as a catheter and vacuum or hydrodynamic draw tubing 104 pass through complementary openings 106 and 108, respectively formed in the cap 102. The tubing 104 provides fluid communication between a vacuum or hydrodynamic draw source 22 and an inner sleeve 12d. The inner sleeve 12d is characterized by a large and rigid pore matrix 18 in fluid communication to a vacuum source 22 such that the source 22 draws (arrow 22D) tissue fluid and fibroblasts 21 into the sleeve 12d. Sleeve 12d has a surface 24 that is optionally nanotextured to promote fibroblast adhesion. The surface 24 is optionally decorated with a pattern of contoured cell-conveying channels. It is appreciated that inner sleeve 12d optionally includes matrix 26 thereover, a coating substance 27, or a combination thereof. The coating 27 is appreciated to need not cover the entire surface 24. The tissue contacting surface 29 of substance 27 is optionally nanotextured. A flange 112 is provided to stabilize the implanted device 100 within the subcuteanous layer S. A flange 112 is constructed from materials and formed by methods conventional to the art. For example, those detailed in U.S. Pat. Nos. 4,634,422; 4,668,222; 5,059,186; 5,120,313; 5,250,025; 5,814,058; 5,997,524; and 6,503,228.

U.S. application Ser. No. 15/555,952 assigned to the assignee of this application discloses a modular external interface that includes a main body with an aperture configured to form a collar seal about an external neck portion of a skin penetrating appliance, such as the PAD 100 of FIG. 1, where a slit extends outward from the aperture. A portal is configured for insertion of a vacuum tube is on the main body, where the portal is in fluid communication with a vacuum channel on a bottom side of the main body. A foam layer is positioned under the main body, and at least one driveline inserted through the aperture and into the appliance. The modular external interface provides additional mechanical stability to an implanted PAD so as to speed healing around a semi-permanent implanted PAD, as well as connection points for vacuum lines and at least one drive line for the insertion of medical devices.

FIGS. 2A-2C illustrate the aforementioned modular external interface housing 200 coupled to the PAD 100 of FIG. 1. The modular external interface 200 forms a collar about the neck 110 of the PAD 100 with the main body 216 with a locking feature 218, such as a male extension that engages a female receptacle or cavity as a mechanical overlap connection. In a specific embodiment the main body 216 is made of silicone. As best shown in FIG. 3, the collar seal between the main body 216 and the neck 110 of the PAD 100 forms a hermetic seal with a gasket 230, which in a specific embodiment is a flexible gasket integrated into the main body 216. In a specific embodiment the gasket 230 may be a floating gasket. The stabilization of the PAD 100 within the skin to form a germ-free barrier requires subject cells to grow onto the neck surfaces 16 of the PAD 100 adjacent to the subject's epidermis E. The neck surface region 16 is adapted to promote growth of autologous fibroblast cells thereon. A suitable exterior side surface substrate for fibroblast growth is a nanotextured polycarbonate (LEXAN®).

The modular external interface 200 is secured and sealed to an outer layer of a patient's skin with a medical dressing. In a specific embodiment the medical dressing is a preform patterned and shaped to conform to the exterior of the modular external interface 200. In a specific embodiment the medical dressing preform may be in two halves (212, 214) that overlap. In a specific embodiment the medical dressing preform may be transparent. In a specific embodiment the medical dressing preform may be made of Tegaderm^{TM} manufactured by Minnesota Mining and Manufacturing Company.

The modular external interface 200 has a central opening adapted at least one drive line 220 for insertion into a PAD, and a portal 224 for a vacuum line 222. As best shown in FIG. 3 a skin protection layer 228 and a foam disc 226 are positioned in the interior of the modular external interface 200.

U.S. application Ser. No. 15/125,273 assigned to the assignee of this application discloses the measurement and monitoring of wound hermaticity of a patient with one or more sensors. The one or more sensors that measure parameters that correlate to a degree of wound hermaticity are incorporated into the design of a percutaneous skin access device (PAD), a bone anchor, a wound dressing, or a bandage. The degree of wound hermaticity is related to impedance measurements performed on the patient's skin, via measurements of humidity in a vacuum line to the PAD or the bone anchor, or via measurements of local tissue oxygenation in the immediate vicinity of the PAD or the bone anchor interface with the patient's skin. The hermaticity measurement parameters are communicated by wired or wireless connection to a computing or a communication device for immediate or remote monitoring.

While there have been many advances in skin penetrating appliance designs for preventing infection at the site of skin access, there continues to be a need for improved external interfaces for implanted appliances. In particular, the myriad of intravenous infusion lines, vacuum lines, and monitoring lines that connect to the PAD and other inserted instruments tend to get tangled, interfere with patient comfort and movement, and are potentially difficult for health care workers to change and maintain.

Thus, there exists a need for improved and integrated multi-lumen tubing for intravenous infusion lines, vacuum lines, and monitoring lines for attachment to percutaneous access devices.

### SUMMARY OF THE INVENTION

An integrated multi-lumen tubing includes two or more lumens. A webbing is provided between each of said two or more lumens and holding the two or more lumens together as a ribbon. A set of terminations are provided at opposing ends of each of the two or more lumens. A sheath can also be provided surrounding the two or more lumens.

A system for supply of intravenous fluid and vacuum to a patient includes the aforementioned integrated multi-lumen tubing. An intravenous bag or bottle is provided that is in fluid communication with an infusion pump. The integrated multi-lumen tubing connects the infusion pump and a vacuum pump to a percutaneous access device (PAD).

A method of using said integrated multi-lumen tubing to delivery simultaneously two or more inputs into a subject in need thereof is also provided.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a prior art, partial cutaway view of a flanged percutaneous access device (PAD) with relative dimensions of aspect exaggerated for visual clarity;

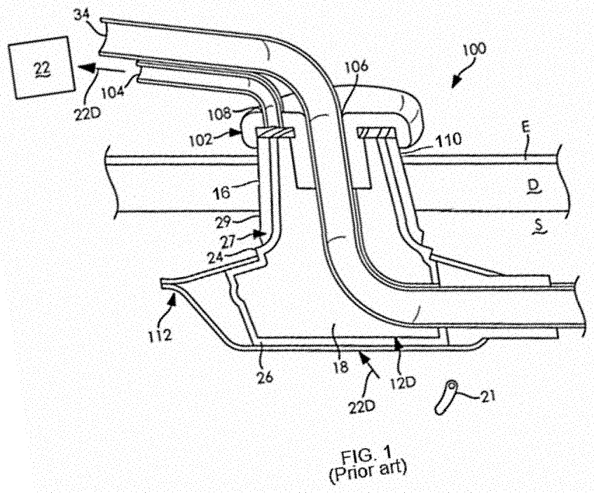

FIGS. 2A-2C are prior art perspective views of a modular external interface seal for a PAD appliance;

FIG. 3 illustrates a side cross sectional view of FIG. 2C;

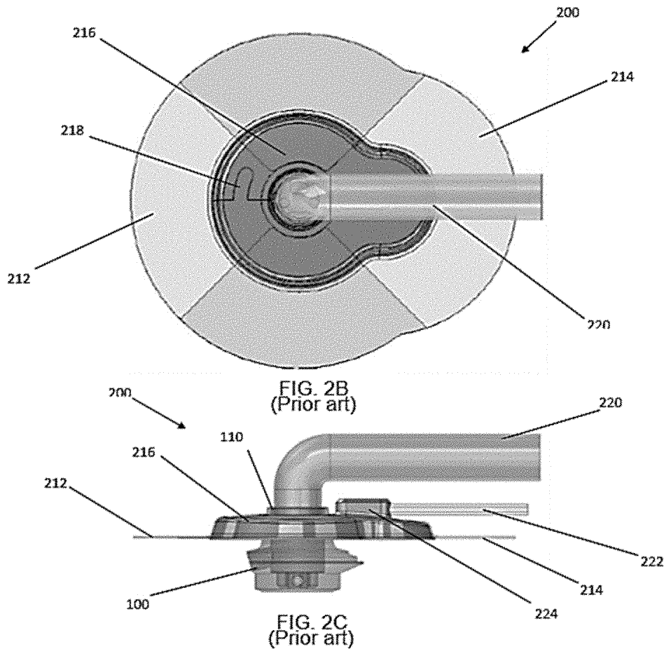

FIG. 4 illustrates web linked integrated multi-lumen tubes for an intravenous infusion line and a vacuum line for attachment to percutaneous access devices according to an embodiment of the invention;

FIG. 5 illustrates web linked integrated muti-lumen tubes for intravenous infusion lines, vacuum lines, and monitoring lines for attachment to percutaneous access devices according to an embodiment of the invention;

FIG. 6 illustrates a sheath covering integrated muti-lumen tubes for an intravenous infusion line and a vacuum line for attachment to percutaneous access devices according to an embodiment of the invention;

FIG. 7 illustrates a system for suppling intravenous fluids and a vacuum via an embodiment of the integrated muti-lumen tubes to the modular external interface seal of FIG. 2 for PAD appliances; and

FIG. 8 illustrates a system with a collocated infusion pump and vacuum pump for suppling intravenous fluids and a vacuum, respectively via an embodiment of the integrated muti-lumen tubes to the modular external interface seal of FIG. 2 for PAD appliances.

### DETAILED DESCRIPTION OF THE INVENTION

The present invention has utility as a system and method for integrated multi-lumen tubing for intravenous infusion lines, vacuum lines, and monitoring lines for attachment to percutaneous access devices. The integration of the intravenous infusion lines, vacuum lines, and monitoring lines that connect to the PAD and other inserted instruments organizes the myriad of intravenous infusion lines, vacuum lines, and monitoring lines that connect to the PAD and other inserted instruments that tend to get tangled, interfere with patient comfort and movement, and are potentially difficult for health care workers to change and maintain.

While embodiments of the integrated multi-lumen tubing are depicted in the accompanying figures as being used with an embedded percutaneous access device (PAD), it is appreciated that it is applicable to a variety of such implant appliances including a catheter, a PICC line, an IV, a Steinman pin, and a Kirschner wire. Embodiments of the integrated muti-lumen tubing provide for the hermaticity in the vicinity of the skin-appliance (PAD) interface with fluid exudate or transudate egres sing from the vicinity of the skin-PAD interface via a vacuum or low pressure tube.

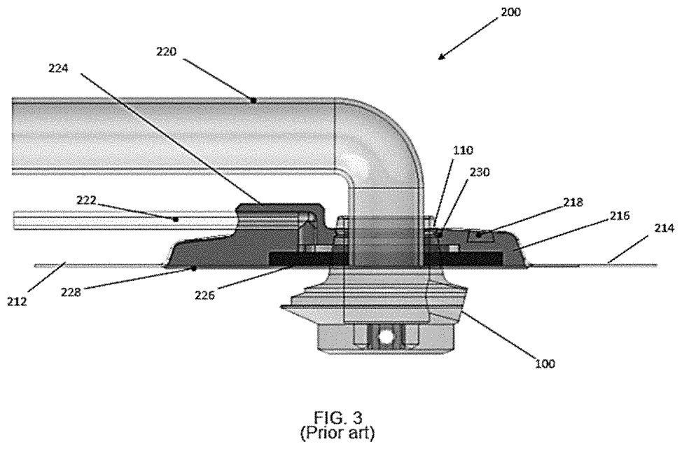

Referring now to the figures, FIG. 4 illustrates an inventive embodiment of a ribbon cable 300 with a webbing 306 that links two or more lumens or tubes for an intravenous infusion line 302 and a vacuum line 304 for attachment to percutaneous access devices. The ribbon cable 300 is generally made of a clear (transparent or translucent) anti-kink material so fluids being transported are visible, as well as to make obvious if any obstructions occur. Non-limiting examples of materials for the lumens or tubes include synthetic polymers such as polytetrafluoroethylene (PTFE), polyvinyl plastic (PVC), polyethylene, or polypropylene plastic. PVC tubing is softened with plasticizers to make it flexible. The webbing 306 is made so that the web may be teared apart to separate the lumens (302, 304) at the terminating ends to facilitate connections. The terminations 308 may be color coated or keyed to avoid improper connections.

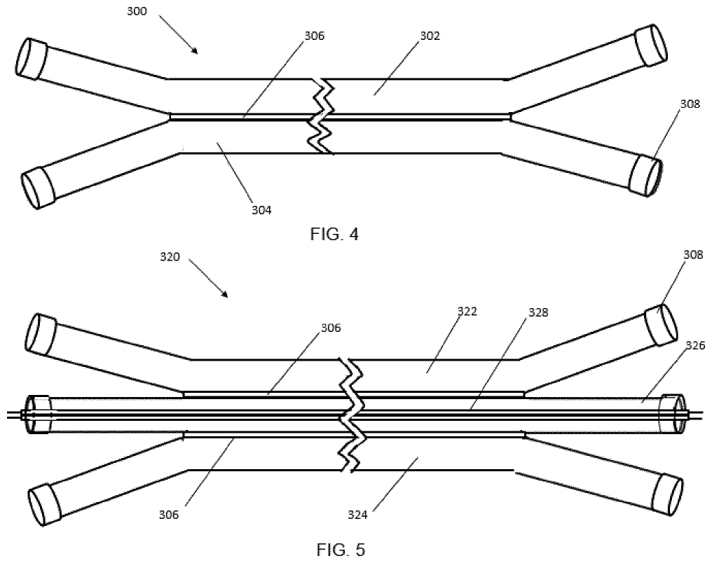

FIG. 5 illustrates an inventive embodiment of an expanded version 320 of web linked integrated muti-lumen tubes for an intravenous infusion line 322, a vacuum line 324, and a monitoring line 326 for wound healing measurements for attachment to percutaneous access devices. A set of insulated wires 328 may be also be attached to the monitoring line 326 for transmitting impedance measurements. Measured parameters that correlate to a degree of wound hermaticity may be incorporated into the design of a percutaneous skin access device (PAD), a bone anchor, a wound dressing, or a bandage. The degree of wound hermaticity is related to impedance measurements performed on the patient's skin, via measurements of humidity in a vacuum line 324 to the PAD, or via measurements of local tissue oxygenation in the immediate vicinity of the PAD as read via monitoring line 326. The hermaticity measurement parameters may be communicated by the wire 328 to a computing or a communication device for immediate or remote monitoring.

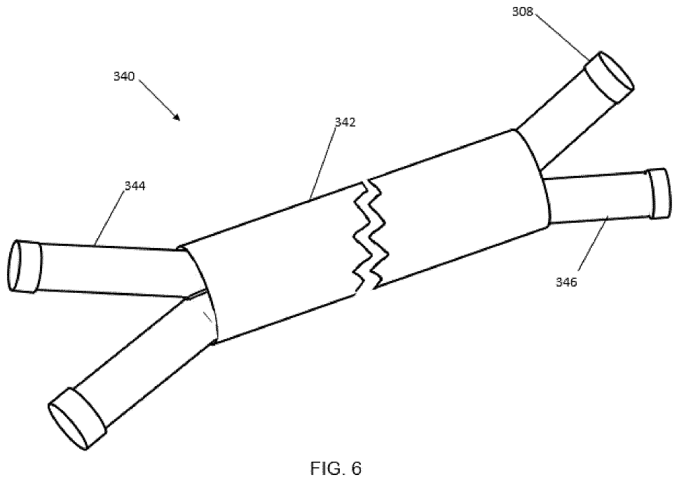

FIG. 6 illustrates an inventive embodiment of a cable 340 with a sheath 342, which is generally clear but is shown as opaque for illustrative clarity, covering integrated muti-lumen tubes for an intravenous infusion line 344 and a vacuum line 346 for attachment to percutaneous access devices. The sheath 342, which is flexible, holds the lumens together and avoids entanglements. It is appreciated that while only two lumens are shown, more than two lumens may be enclosed in the sheath 342 in accordance with embodiments of the invention.

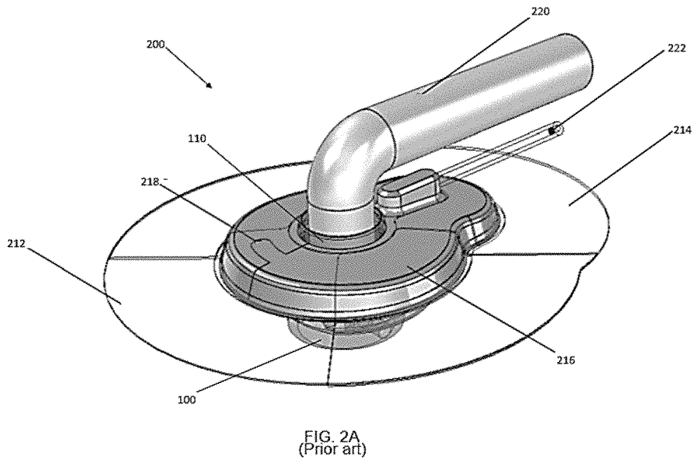

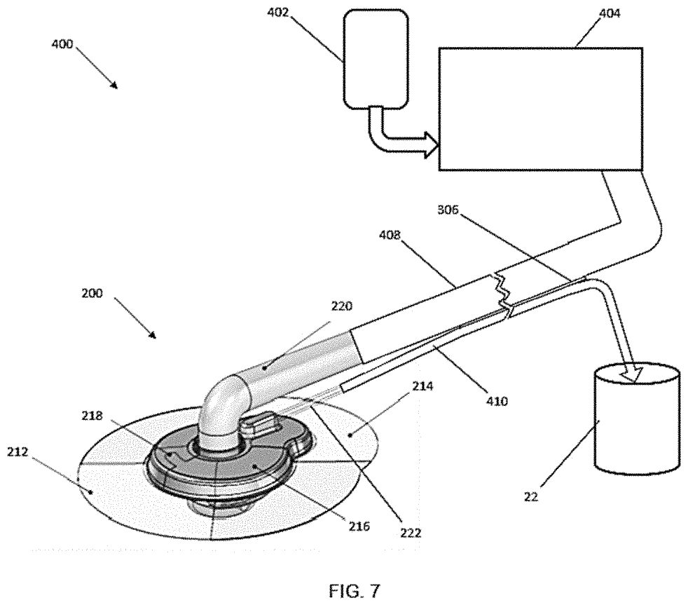

FIG. 7 illustrates a system 400 for suppling intravenous (IV) fluids and a vacuum via an embodiment of the integrated muti-lumen tubes to the modular external interface seal 200 of FIG. 2 for PAD appliances. An intravenous bag or bottle 402 is shown supplying an infusion pump 404. The IV fluids are supplied via an infusion line 408 to the driveline 220 of the PAD 200. A vacuum line 410 attached to the infusion line 408 with web 306 terminates in a vacuum pump 22 and the vacuum line 222 of the PAD 200.

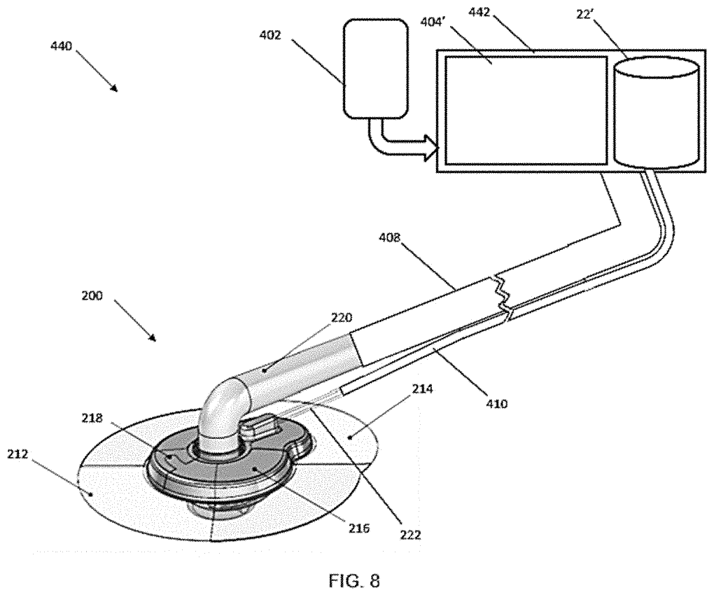

FIG. 8 illustrates a system 440 with an integrated device 442 with a collocated infusion pump 404′ and vacuum pump 22′ for suppling intravenous fluids and a vacuum, respectively via an embodiment of the integrated muti-lumen tubes to the modular external interface seal of FIG. 2 for PAD appliances. An intravenous bag or bottle 402 is shown supplying an infusion pump 404′. The IV fluids are supplied via an infusion line 408 to the driveline 220 of the PAD 200. A vacuum line 410 attached to the infusion line 408 with web 306 terminates in a vacuum pump 22 and the vacuum line 222 of the PAD 200.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. An integrated multi-lumen tubing comprising:
two or more lumens;
a webbing between each of said two or more lumens, said webbing holding the two or more lumens together as a ribbon; and
a set of terminations at opposing ends of each of said two or more lumens.

2. The integrated multi-lumen tubing of claim 1 wherein said two or more lumens comprise an intravenous infusion line and a vacuum line.

3. The integrated multi-lumen tubing of claim 2 wherein said two or more lumens further comprises a monitoring line for wound healing.

4. The integrated multi-lumen tubing of claim 2 wherein said two or more lumens further comprises a set of insulated wires for transmitting impedance measurements.

5. The integrated multi-lumen tubing of claim 1 wherein said two or more lumens are transparent or translucent.

6. The integrated multi-lumen tubing of claim 1 wherein said lumens are made of synthetic polymers such as polytetrafluoroethylene (PTFE), polyvinyl plastic (PVC), polyethylene, or polypropylene plastic.

7. The integrated multi-lumen tubing of claim 1 wherein said set of terminations is color coded or keyed.

8. The integrated multi-lumen tubing of claim 1 further comprising a sheath covering said two or more lumens.

9. The integrated multi-lumen tubing of claim 8 wherein said two or more lumens comprise an intravenous infusion line and a vacuum line.

10. The integrated multi-lumen tubing of claim 9 wherein said two or more lumens further comprises a monitoring line for wound healing.

11. The integrated multi-lumen tubing of claim 9 wherein said two or more lumens further comprises a set of insulated wires for transmitting impedance measurements.

12. The integrated multi-lumen tubing of claim 8 wherein said two or more lumens are transparent or translucent.

13. The integrated multi-lumen tubing of claim 8 wherein said lumens and said sheath are made of synthetic polymers such as polytetrafluoroethylene (PTFE), polyvinyl plastic (PVC), polyethylene, or polypropylene plastic.

14. The interface multi-lumen tubing of claim 8 wherein said set of terminations is color coded or keyed.

15. A system for supply of intravenous fluid and vacuum to a patient, said system comprising:
said integrated multi-lumen tubing of claim 1;
an intravenous bag or bottle in fluid communication with an infusion pump;
a vacuum pump; and
wherein said integrated multi-lumen tubing connects said infusion pump and said vacuum pump to a percutaneous access device (PAD).

16. A method of using said integrated multi-lumen tubing of claim 1 for creating at least two of an intravenous infusion line, a vacuum line, or monitoring line to a subject in need thereof.
